(19) United States
(12) Patent Application Publication
(10) Pub. No.: US 2023/0173481 A1
(43) Pub. Date: Jun. 8, 2023

(54) METHOD OF FECAL SAMPLE PREPARATION FOR AUTOMATED IMAGE ANALYSIS

(71) Applicant: Parasight System Inc., Lexington, KY (US)

(72) Inventors: Pawel Slusarewicz; Christopher Patrick Mills, McLean, VA (US); David James Schmeling, San Diego, CA (US); Christopher Schroeder, San Diego, CA (US)

(21) Appl. No.: 17/990,137

(22) Filed: Nov. 18, 2022

Related U.S. Application Data
(60) Provisional application No. 63/285,657, filed on Dec. 3, 2021.

(51) Int. Cl. B01L 3/00; G01N 1/40; G01N 1/38

(52) U.S. Cl. CPC B01L 3/50215; G01N 1/38; G01N 1/4077; B01L 2200/16; B01L 2300/0681; B01L 2300/0832; G01N 2001/4088

(57) ABSTRACT

A method of analyzing a fecal sample containing parasitic material and non-parasitic material. The fecal sample is first deposited into a carrier containing a flotation medium. The carrier is then shaken to disperse the fecal sample in the flotation medium. The fecal sample is then allowed to settle under gravity or by centrifugation. A filter device is then inserted into the carrier to separate the parasitic material from the non-parasitic material and to collect the parasitic material. The collected parasitic material is then poured into a capture filter for analysis.

### CROSS-REFERENCE TO RELATED APPLICATIONS

The present application claims priority to U.S. Provisional Pat. Application No. 63/285,657, filed Dec. 3, 2021, the entirety of which is incorporated herein by reference.

### TECHNICAL FIELD

The present disclosure generally relates to an apparatus and methods of utilizing the apparatus to separate ova from fecal particles prior to the ova being analyzed.

### BACKGROUND

Infection with gastrointestinal parasites is frequently diagnosed through the detection of parasite ova and/or oocysts in the host’s feces. Traditionally this has been achieved by manual examination of the feces using a microscope. More recently, however, a number of systems have been developed wherein parasites are detected automatically using computer vision algorithms. All but one of these methods require the physical separation of ova from the bulk of the denser fecal particulates as well as high-optical magnification/low field-of-view imaging to provide the fidelity to identify the ova computationally.

Separation is typically achieved using a dense floatation medium (FM) and is conducted to help differentiate the parasitic products from the background material. Since parasitic products are less dense than most other fecal components, they tend to float in FMs while the bulk of the remaining fecal matter sinks. In some automated methods, a slurry of feces in FM is placed in a chamber and separation occurs under gravity. The depth of the chamber is sufficient to allow a high-power, shallow depth-of-field imaging system to focus on the floating eggs while defocusing the sunken fecal material. This differential focusing facilitates the identification of the ova; this would otherwise be problematic in the presence of the other fecal material, which represents the vast bulk of the feces.

Another automated method uses either gravity or centrifugation of a slurry in a tube to accelerate separation and to concentrate ova at the surface of the liquid. The ova are then harvested from the surface of the liquid, placed on a glass slide, overlaid with a coverslip and imaged at high magnification. The high magnification used by these systems requires the capture of multiple fields in order to image the entire sample, which is a time-consuming process.

One advantage of centrifugation is the ability to analyze a greater amount of feces, thereby potentially increasing test sensitivity. In fact, several manual methods exist where centrifugation is used and the ova collected on a coverslip for examination. In one case, a large amount of a slurry is placed in a tube to form a meniscus onto which the coverslip is overlaid prior to centrifugation. In other cases, tubes are not filled completely prior to centrifugation but then subsequently topped off with FM to form a meniscus, and then a coverslip is overlaid. In this case the sample is allowed to stand for sufficient time to allow the final floatation of the ova to the coverslip to occur under gravity.

The advantages of direct coverslip methods are a slight reduction in processing time (because they do not require the final gravitational floatation step), and potentially better egg recovery due to an increase in adhesion of ova to the coverslip because of the high g-force generated by the centrifuge. However, their disadvantage lies in the possibility of detachment of the coverslip during centrifugation resulting in sample loss and contamination of the centrifuge.

In contrast, the automated centrifugal method does not collect eggs directly onto the coverslip. Instead, ova are collected using a sample loop pressed onto the surface of the liquid post-centrifugation. Since this is a variable process (since only a tiny amount of liquid is collected, which is likely does not collect the majority of the floated ova), this method of sample collection could have negative repercussions on test performance parameters such as sensitivity and precision.

One automated test differs markedly from the methods described above in that it does not require separation of the parasitic products from the feces because the ova are fluorescently labeled with an ova-specific dye and imaged in fluorescence mode in order to aid in differentiating the ova from the remainder of the feces. Furthermore, instead of imaging ova under a coverslip or in a specialized chamber, the eggs are trapped on a mesh filter with a sufficiently small pore size to prevent eggs from passing through. Staining and washing of the sample is conducted on this mesh prior to imaging.

One advantage of this method is that the mesh serves as a convenient method to quantitatively harvest and concentrate ova from the slurry to the imaging area, which is not the case with the other methods described above. While this test can be performed without separation of the fecal components by density, its sensitivity is limited to the amount of material that can be loaded onto the filter membrane prior to it becoming clogged with fecal particles. Furthermore, large amounts of fecal debris can obscure eggs on the filter and prevent their detection. Additionally, some fecal particles can be autofluorescent (particularly in carnivore feces) and thus minimizing their presence can reduce the possibility of misidentification.

Therefore, there is a need in the art for a device and method of using the device to maximize the recovery of ova while minimizing contamination with fecal particles from a fecal sample prior to the sample being analyzed.

### SUMMARY

An embodiment of the present disclosure teaches a method of analyzing a fecal sample containing parasitic material and non-parasitic material comprising: depositing the fecal sample into a carrier; depositing a flotation medium into the carrier; shaking the carrier to disperse the fecal sample in the flotation medium; allowing the fecal sample to settle under gravity or by centrifugation; inserting a filter device into the carrier to separate the parasitic material from the non-parasitic material and to collect the parasitic material; and pouring the parasitic material into a capture filter for analysis.

Another embodiment provides a method as in any embodiment above, wherein the filter device comprises a tube and a mesh filter attached to an end of said tube.

Another embodiment provides a method as in any embodiment above, wherein the end of the tube of the filter device that the mesh filter is attached to is the end inserted into the carrier.

Another embodiment provides a method as in any embodiment above, wherein the mesh filter has a pore size of between about 20 microns and about 1,000 microns.

Another embodiment provides a method as in any embodiment above, wherein the filter device further comprises an O-ring, a gasket, or a flange secured within a groove in the tube adjacent the mesh filter and one or more notches on an end of the tube opposite the mesh filter.

Another embodiment provides a method as in any embodiment above, wherein the filter device further comprises an O-ring, a gasket, or a flange slid over a step on the end of the tube adjacent the mesh filter and one or more notches on an end of the tube opposite the mesh filter.

Another embodiment provides a method as in any embodiment above, wherein the filter device is inserted at a depth of between 0.25 and 5 inches below the surface of the liquid in the carrier.

Another embodiment provides a method as in any embodiment above, wherein the step of collecting the fecal sample is collected with a sample collection tool containing a plunger.

Another embodiment provides a method as in any embodiment above, wherein the step of collecting includes drawing back the plunger to a metered position on the sample collection tool and filling the sample collection tool with the fecal sample; and wherein the step of depositing the fecal sample includes depressing the plunger of the sample collection tool such that the fecal sample is deposited within the carrier.

Another embodiment provides a method as in any embodiment above, further comprising depositing a dispersion aid into the carrier.

Another embodiment provides a method as in any embodiment above, further comprising the step of centrifuging the carrier after the step of shaking.

Another embodiment provides a method as in any embodiment above, further comprising the step of placing the capture filter within a reagent dispensing unit for the dispensing of reagents that will turn the collected parasitic material into fluorescent parasitic material.

Another embodiment provides a method as in any embodiment above, further comprising the step of placing the capture filter within an imaging unit for imaging of the fluorescent parasitic material for analysis of the fluorescent parasitic material.

Another embodiment provides a method as in any embodiment above, wherein the flotation medium has a density of between about 1.1 g/mL and about 1.2 g/mL.

An additional embodiment of the present disclosure provides a kit for use in analyzing a fecal sample comprising: a filter device comprising a tube and a mesh filter attached to an end of said tube; a sample collection tool; and a fecal sample carrier.

Another embodiment provides a kit as in any embodiment above, wherein the mesh filter of the filter device has a pore size of between about 20 microns and about 1,000 microns.

Another embodiment provides a kit as in any embodiment above, wherein the filter device further comprises an O-ring, a gasket, or a flange secured within a groove in the tube adjacent the mesh filter and one or more notches on an end of the tube opposite the mesh filter.

Another embodiment provides a kit as in any embodiment above, wherein the filter device further comprises an O-ring, a gasket, or a flange slid over a step on the end of the tube adjacent the mesh filter and one or more notches on an end of the tube opposite the mesh filter.

Another embodiment provides a kit as in any embodiment above, further comprising a dispersion aid placeable within the fecal sample carrier.

Another embodiment provides a kit as in any embodiment above, wherein the sample collection tool contains a plunger.

### BRIEF DESCRIPTION OF THE DRAWINGS

The present disclosure will be more readily understood from a detailed description of some example embodiments taken in conjunction with the following figures:

FIG. 1A is an exploded view of one embodiment of the ova filter device of the present disclosure;

FIG. 1B is an exploded view of one embodiment of the ova filter device of the present disclosure;

FIG. 2 shows a plain view of the ova filter device of FIG. 1 prior to insertion into a carrier device containing a dispersion aid;

FIG. 3 shows an isometric view of a sample collection tool of the present disclosure;

FIG. 4 shows a plain view of the ova filter device of FIG. 1 as inserted into a carrier device containing a fecal sample pellet;

FIG. 5 shows a plain view of a capture filter, a reagent dispensing unit, an imaging unit, and a tablet computer 60 as utilized in methods of the present disclosure;

FIG. 6A shows an image of a sample collected without the use of the ova filter device of the present disclosure;

FIG. 6B shows an image of a sample collected utilizing the ova filter device of the present disclosure;

FIG. 7A shows an image of a sample collected utilizing a flotation medium having a density of 1.2 g/mL; and

FIG. 7B shows an image of a sample collected utilizing a flotation medium having a density of 1.1 g/mL.

### DETAILED DESCRIPTION OF ILLUSTRATIVE EMBODIMENTS

Various non-limiting embodiments of the present disclosure will now be described to provide an overall understanding of the principles of the structure, function, and use of the apparatuses, systems, methods, and processes disclosed herein. One or more examples of these non-limiting embodiments are illustrated in the accompanying drawings. Those of ordinary skill in the art will understand that systems and methods specifically described herein and illustrated in the accompanying drawings are non-limiting embodiments. The features illustrated or described in connection with one non-limiting embodiment may be combined with the features of other non-limiting embodiments. Such modifications and variations are intended to be included within the scope of the present disclosure.

Reference throughout the specification to “various embodiments,” “some embodiments,” “one embodiment,” “some example embodiments,” “one example embodiment,” or “an embodiment” means that a particular feature, structure, or characteristic described in connection with any embodiment is included in at least one embodiment. Thus, appearances of the phrases “in various embodiments,” “in some embodiments,” “in one embodiment,” “some example embodiments,” “one example embodiment,” or “in an embodiment” in places throughout the specification are not necessarily all referring to the same embodiment. Furthermore, the particular features, structures or characteristics may be combined in any suitable manner in one or more embodiments.

A “computer,” “computer system,” “host,” “server,” or “processor” can be, for example and without limitation, a processor, microcomputer, minicomputer, server, mainframe, laptop, personal data assistant (PDA), wireless e-mail device, cellular phone, pager, processor, fax machine, scanner, or any other programmable device configured to transmit and/or receive data over a network. Computer systems and computer-based devices disclosed herein can include memory for storing certain software modules used in obtaining, processing, and communicating information. It can be appreciated that such memory can be internal or external with respect to operation of the disclosed embodiments. The memory can also include any means for storing software, including a hard disk, an optical disk, floppy disk, ROM (read only memory), RAM (random access memory), PROM (programmable ROM), EEPROM (electrically erasable PROM) and/or other computer-readable media. Non-transitory computer-readable media, as used herein, comprises all computer-readable media except for a transitory, propagating signals.

In various embodiments disclosed herein, a single component can be replaced by multiple components and multiple components can be replaced by a single component to perform a given function or functions. Except where such substitution would not be operative, such substitution is within the intended scope of the embodiments.

Described herein are example embodiments of apparatuses, systems, and methods for separating ova from fecal particles prior to the ova being analyzed within an imaging and analysis device(s). Routine fecal egg count (FEC) and FEC reduction (FECR) analyses are an integral part of parasite management strategies in equids and in small ruminants such as cows, sheep, and goats. It has also become an important analysis to undertake on household animals such as dogs and cats since the evolution and spread of anthelmintic resistance in Ancylostoma (hookworms). Parasite load has been shown to adversely affect productivity in livestock. While many cat and dog parasites are zoonotic, and so the development of anthelmintic resistance represents a growing concern to the agricultural and household pet industries.

Numerous methods are available for conducting FECs and FECRs. Most of the tests are based on the lower density of ova relative to the non-parasitic elements of the bulk feces, and ova are separated by flotation when a fecal sample is suspended in a dense medium. Most methods utilize a flotation medium having a density between 1.2 and 1.3 g/mL. This separation facilitates the identification and counting of the ova by reducing the background produced by the non-parasitic components of the feces.

Embodiments of the present disclosure will be discussed in relation to a method that utilizes a single low-magnification image with a broad field of view that can nevertheless be analyzed computationally. However, any method for conducting FECs and FECRs that can benefit from the apparatuses and methods discussed below are considered to be covered by the present disclosure.

The method of taking a single low-magnification image with a broad field of view is accomplished by fluorescently labeling eggs. One such method utilizes the presence of chitin in their shells by using a derivatized recombinant chitin-binding protein. Imaging in fluorescence mode facilitates the production of high contrast, low background images with sufficient fidelity for automated identification even at low magnification. As a result, only a single image is needed for each sample, and the number of eggs present can be counted in less than a minute, while a four-minute sample preparation and processing procedure is simplified by means of an automated device(s).

In one or more embodiments of the present disclosure eggs of different strongylid genera can be differentiated by fluorescent staining using other proteins ligands (such as lectins). The type of parasitic material analyzed include one or more of the following: members of the genus Cooperia, members of the genus Ostertagia, members of the genus Trichostrongylus, members of the genus Haemonchus contortus and members of the genus Bunostomum,

While these tests can be performed without separation of the fecal components by density, their sensitivity is limited to the amount of material that can be loaded onto a filter membrane prior to it becoming clogged with fecal particles. Furthermore, large amounts of fecal debris can obscure eggs on the filter and prevent their detection. Additionally, some fecal particles can be auto-fluorescent (particularly in carnivore feces) and thus minimizing their presence can reduce the possibility of misidentification.

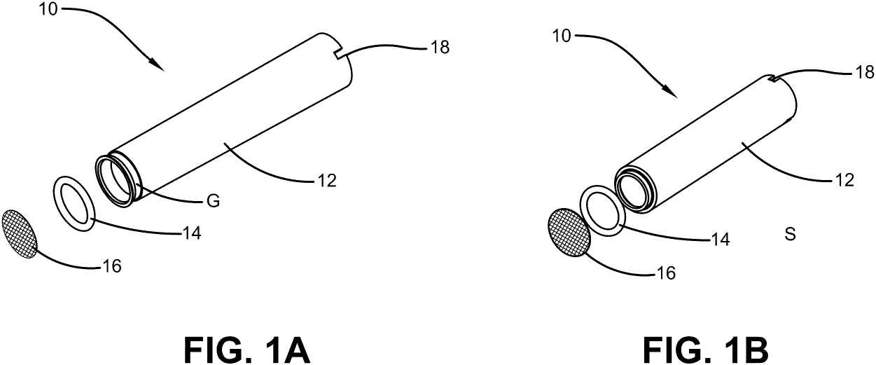

The present disclosure aims to solve this problem by further separating the ova from the fecal particles prior to addition of the slurry to a mesh egg chamber, thereby increasing the amount of feces that can be analyzed and minimizing the number of potentially interfering/clogging particles. To do this, an ova filter device 10 as shown in FIGS. 1A and 1B is utilized. The ova filter device 10 includes an egg separator tube 12, an O-ring or other type of gasket 14, and a mesh filter 16. The mesh filter 16 is attached to the egg separator tube 12. In one or more embodiments, the means of attachment include the use of an adhesive, by heat staking or by sonic welding. In one or more embodiments, the ova filter device 10 also contains one or more notches 18 on the egg separator tube 12 at the opposite end of the tube 12 to the mesh filter 16. The one or more notches 18 allow air to escape when the ova filter device 10 is pressed into a sample tube. The length of the egg separator tube 12 needs to be long enough so as to reach below the level of the least-floated ovum/oocyst, but ideally short enough so as not to disturb the sedimented fecal material when the device 10 is pressed into a sample tube. In one or more embodiments, the length of the egg separator tube 12 is between about 2 and about 4 inches. In one embodiment, the length of the egg separator tube 12 is 3 inches. In another embodiment, the device 10 contains a flange (not shown) instead of the O-ring 14. If present, the flange serves the same purpose as the O-ring 14, which is to prevent loss of sample between the sample tube and the tube 12 of device 10.

FIG. 1A shows an embodiment of the ova filter device 10 wherein the O-ring 14 is placed within a groove G adjacent the end of the ova filter device 10. FIG. 1B shows an embodiment of the ova filter device 10 wherein instead of a groove G adjacent an end of the ova filter device, the end of the ova filter device is stepped such that a step S is created. In the embodiment shown in FIG. 1B, the O-ring 14 should be slid over the step S prior to the mesh filter 16 being attached to the ova filter device 10.

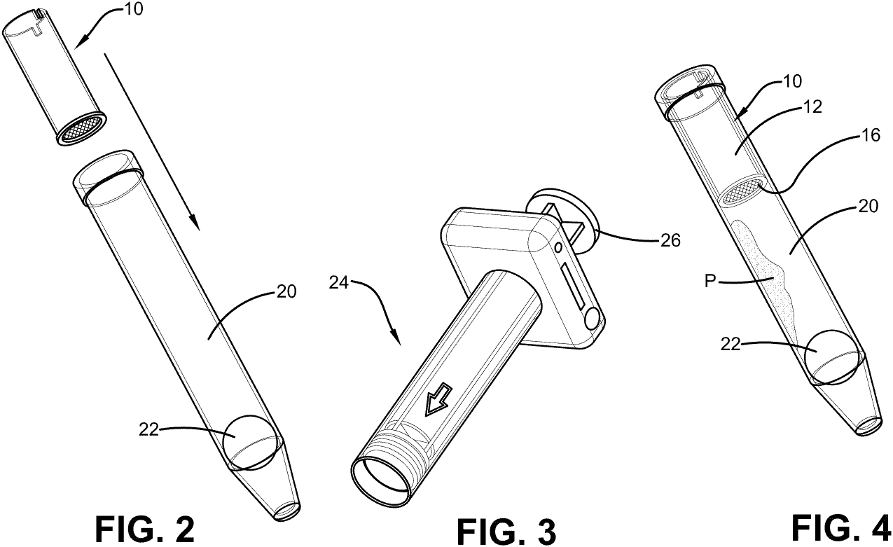

Other components needed for the method of the present disclosure are shown in FIG. 2 and include a sample centrifuge tube 20 and a dispersion aid 22, such as the ball bearing shown. The dispersion aid 22 can either be disposable or reusable. In one or more embodiments, the dispersion aid 22 incorporates fins or blades to aid in sample dispersion. In one or more embodiments, the centrifuge tube 20 is a 15 mL tube. In one or more embodiments, the centrifuge tube 20 can be supplied to the end user preloaded with between 5 and 14 mL of a flotation medium, such as, but not limited to sodium nitrate, sodium chloride, sugar (such as sucrose) a sugar salt mix, magnesium sulfate or zinc sulfate. In one embodiment, the tube 20 is preloaded with 10 mL of a flotation medium. If not preloaded, between 5 and 14 mL of a flotation medium should be added to the centrifuge tube 20 prior to any fecal sample being added to the tube 20. In another embodiment, the tube is preloaded with the dispersion aid either with or without the floatation medium.

To analyze a fecal sample containing parasitic material and non-parasitic material according to one or more embodiments of the present disclosure, a fecal sample will need to be placed in the centrifuge tube 20. In one or more methods of the present disclosure, the fecal sample is prepared by utilizing a sample collection tool 24 as shown in FIG. 3. The sample collection tool 24 gathers a small fecal sample from the collected fecal sample, and then the small fecal sample is deposited into the tube 20. In one or more embodiments, the sample collection tool 24 contains a plunger 26 that can be utilized to assist in depositing the small fecal sample into the tube 20. To fill the sample collection tool 24, the plunger 26 should be pulled back, which will meter the collection. Then, once the tool 24 is filled with a fecal sample, the plunger 26 can be depressed for easy ejection of the sample from the tool 24 into the tube 20. Once the sample has been added to the tube 20, the dispersion aid 22 can then be added to the tube 20, and the tube 20 is then capped with any conventional tube cap. It is also contemplated that the dispersion aid 22 can be added to the tube 20 prior to introduction of the fecal sample.

Once capped, the centrifuge tube 20 should be vigorously shaken to begin the process of mixing the fecal sample with the flotation medium. This shaking will also assist in breaking apart the fecal sample. Once shaken, in some embodiments of the present disclosure, the centrifuge tube 20 is then added to a centrifuge. The tube 20 should then be centrifuged with a counter balance for a time period of about between about 30 seconds and 5 minutes at a speed of between about 1,000 RPM and 8,000 RPM. In one embodiment, the tube 20 is centrifuged for 2 minutes at 2,400 RPM. In other embodiments, centrifugation is not needed, and after the period of shaking, the contents of the tube 20 are allowed to just settle on their own for a time period of between about 5 minutes and 60 minutes. However, without centrifugation, the results of the method will take longer to collect due to the prolonged standing time.

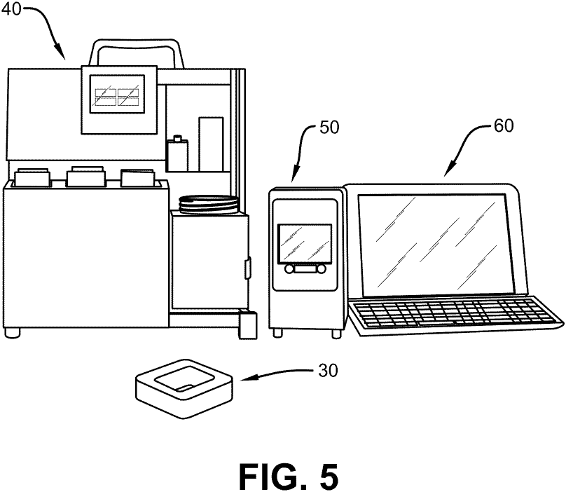

Either after a standing period or the end of the centrifugation, the tube 20 is then uncapped. The tube 20 will then have a pellet of matter P present at the bottom of tube 20. Next, the ova filter device 10 is pressed into the tube 20. The ova filter device 10 should be inserted with the mesh filter 16 entering the tube 20 first. The ova filter device 10 should be placed at a depth of between about 0.25 and about 6 inches below the surface of the liquid within the tube 20. In one or more embodiments, when using an imaging system as discussed above, once the ova filter device 10 is in place, the sample captured within the tube 12 is then poured onto a capture filter 30, such as an egg chamber filter as shown in FIG. 5. In one or more embodiments, the egg chamber 30 is then placed within a reagent dispensing unit 40 and then an imaging unit 50 as shown in FIG. 5. Although the reagent dispensing unit 40 and the imaging unit 50 are shown in two different housings, it is also contemplated that both units can be housed within one singular unit. The reagent dispensing unit 40 will dispense various reagents that allow for ova and oocysts to fluoresce, which will then allow for the imaging unit 50 to take better images for the ova and oocysts. The images taken by the imaging unit 50 can then be viewed on any computing device, such as the computing device 60 shown in FIG. 5. The computing device 60 will also run an egg-counting algorithm in one or more embodiments.

In another embodiment, the ova filter device 10 can be placed into the tube 20 prior to shaking or after shaking but before the optional centrifugation. In this embodiment, large fecal particles are forced downwards while the ova and oocysts pass through the mesh filter 16 into the device 10 during shaking and the optional centrifugation. During shaking and the optional centrifugation, any fecal material pressed against the mesh filter 16 will sink downwards, thereby unclogging the mesh filter 16 and allowing the ova and oocysts below the mesh filter 16 to flow upwards and through the mesh filter.

When the device 10 is pressed into the centrifuge tube 20, the mesh filter 16 separates the bulk fecal matter at the bottom of tube 20 from the liquid containing the floating ova and oocysts at the top of the tube 20. The ova and oocysts float due to the density of the ova and oocysts as compared to the density of the floatation medium within the tube 20. This separation by device 10 will prevent clumps of fecal matter that may dislodge from the pellet P from entering into the capture filter 30 when the sample is poured therein. The pores of the mesh filter 16 therefore need to be sufficiently large enough to allow the ova and oocysts to pass through, but small enough to prevent the liquid below it from flowing through by virtue of the surface tension created by insertion of the device 10 into the tube 20. In other words, the mesh filter 16 does not simply prevent larger fecal particle from passing through, but rather the mesh filter 16 prevents all of the liquid and solid material located beneath the mesh filter 16 from passing through. In one or more embodiments, the mesh filter should have a pore size of between about 80 microns and about 1,000 microns. In the case of samples that contain smaller parasite products such as coccidian oocysts, the pore size can be as low as 20-40 microns.

The purpose of the ova filter device is to separate the bulk feces of the pellet of matter P at the bottom of the tube 20 from the liquid (containing floating eggs) above it. This will prevent clumps of feces that may dislodge from the pellet P from entering the capture filter 30. The pores of the mesh filter 16 of the ova filter device 10 therefore need to be sufficiently large to allow ova and oocysts to pass though, but small enough to prevent the liquid below it from flowing through by virtue of the surface tension of the mesh filter 16. That is to say, the purpose of the mesh filter 16 is not simply to prevent larger particles from passing through, but rather to prevent all the liquid and solid beneath it from doing so. In one or more embodiments, the mesh filter 16 should have pore sizes between 20 and 1000 microns. Since, unlike other centrifugal manual or automated methods, the ova do not need to reach the surface of the liquid for harvesting or examination, but rather only need to reach a level above the final level of the inserted ova filter device 10, this reduces the amount of centrifugation time required for the test. The device 10 will also assist in stopping the dispersion aid 22 from falling onto the capture filter 30. In one or more embodiments, if the animal that produced the sample is a ruminant, such as a cow, a sheep, or a goat, the pore size of the capture filter 30 should be between 20 microns and 40 microns, and in on embodiment the pore size of the capture filter is 25 microns. In one embodiment, if the animal that produced the sample is a cat or a dog, the pore size of the capture filter 30 should be between 15 microns and 25 microns, and in one embodiment the pore size of the capture filter is 20 microns. In one or more embodiments, if the animal that produced the sample is an equine, the pore size of the mesh filter 16 should be between 30 microns and 50 microns, and in one embodiment the pore size of the capture filter is 37 microns.

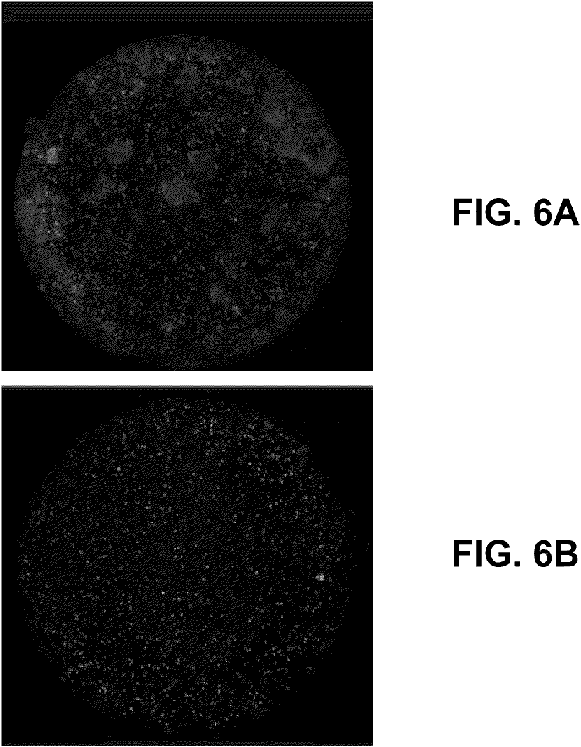

The effectiveness of the device 10 used in the method discussed above in minimizing the presence of non-parasite material in the processed samples is shown in FIGS. 6A and 6B. FIG. 6A shows an image of a sample that was centrifuged, poured directly onto a capture filter, such as capture filter 30, without the use of device 10, dyed with a fluorescent chitin-binding protein, and then imaged. FIG. 6B shows an image produced from the same sample that was centrifuged, filtered with device 10, poured directly onto a capture filter, such as capture filter 30, dyed with a fluorescent chitin=binding protein, and then imaged. While both FIGS. 6A and 6B contain numerous canine hookworm ova (small dots), the image of FIG. 6B in which the sample was filtered with device 10 is characterized by the marked elimination of large, non-parasitic fecal particles which can obscure ova, clog the filter and interfere with the automated analysis.

In one embodiment, the animal feces analyzed is produced by sheep, and the specific parasitic material that is being search for are Haemonchus contortus (HC) ova. HC is a particularly pathogenic helminth that is responsible for the death of significant numbers of lambs annually. Because it is difficult to reliably distinguish between sheep strongylid ova visually, HC infection is commonly diagnosed by coproculture of the fecal sample and microscopic examination of the resulting larvae. Due to the hatching time required, such tasks are time consuming (taking up to 2 weeks) and require analysts with specific training and expertise in larval identification. It has been shown that HC ova can be distinguished from other strongylids by fluorescent staining with the lectin peanut agglutinin (PNA). However such a method has not been widely adopted, perhaps because of the need for a fluorescence microscope.

The fluorescence detection system described above is well suited to the detection and enumeration of HC ova following PNA staining. However, PNA staining is substantially duller than staining with a chitin-binding protein. This requires imaging at significantly higher exposures, which results in overexposure of the residual fecal material, which interferes with the detection of the ova. The ova filter device 10 described here helps to ameliorate this problem to some extent, but not entirely due to the high exposure required to observe the HC ova.

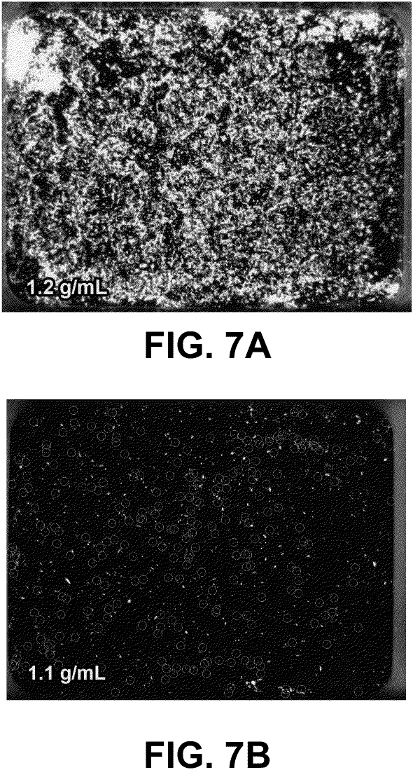

It has been discovered that this particular problem can be solved by using a floatation medium with a significantly lower-than-usual density. As discussed above, the typical density of the floatation medium used for conducting FECs and FECRs have a density between 1.2 and 1.3 g/mL. However, at these higher densities, too much debris is found in sheep samples to reliably detect HC ova. By using a flotation medium of substantially lower density (1.1 g/mL), the amount of fecal debris is vastly lowered and a clean analysis can take place. The results of this are shown in FIGS. 7A and 7B, wherein FIG. 7A shows an image of a sample that utilized a flotation medium of 1.2 g/mL and FIG. 7B shows an image of a sample that utilized a flotation medium of 1.1 g/mL. While both FIGS. 7A and 7B contain numerous HC ova (small dots), the image of FIG. 7B is characterized by the marked elimination of large, non-parasitic fecal particles that can obscure ova and interfere with the automated analysis. In this embodiment, it is also contemplated that the device 10 as discussed above can be utilized in the method. Although the above method was discussed in relation to sheep and HC, it can equally be used for producing cleaner samples in other applications, in conjunction or without the device 10. For example, it is also contemplated that a flotation medium having a density lower than typically used in conducting FECs and FECRs can be utilized to analyze fecal matter of household animals like cats and dogs. However, since cats and dogs can be infected with helminths of the genus Tirichuris, which have a density of approximately 1.15 g/mL, a medium with a density of 1.1 g/mL would therefore be insufficient to float such eggs and it has been discovered that a flotation medium with a density of 1.18 g/mL is more suitable for this application.

The examples discussed herein are examples only and are provided to assist in the explanation of the apparatuses, devices, systems and methods described herein. None of the features or components shown in the drawings or discussed below should be taken as mandatory for any specific implementation of any of these the apparatuses, devices, systems or methods unless specifically designated as mandatory. For ease of reading and clarity, certain components, modules, or methods may be described solely in connection with a specific figure. Any failure to specifically describe a combination or sub-combination of components should not be understood as an indication that any combination or sub-combination is not possible. Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

Having shown and described various versions in the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

What is claimed is:

1. A method of analyzing a fecal sample containing parasitic material and non-parasitic material comprising:
a. depositing the fecal sample into a carrier;
b. depositing a flotation medium into the carrier;
c. shaking the carrier to disperse the fecal sample in the flotation medium;
d. allowing the fecal sample to settle under gravity or by centrifugation;
e. inserting a filter device into the carrier to separate the parasitic material from the non-parasitic material and to collect the parasitic material; and
f. pouring the parasitic material into a capture filter for analysis.

2. The method of claim 1, wherein the filter device comprises a tube and a mesh filter attached to an end of said tube.

3. The method of claim 2, wherein the end of the tube of the filter device that the mesh filter is attached to is the end inserted into the carrier.

4. The method of claim 2, wherein the mesh filter has a pore size of between about 20 microns and about 1,000 microns.

5. The method of claim 2, wherein the filter device further comprises an O-ring, a gasket, or a flange secured within a groove in the tube adjacent the mesh filter and one or more notches on an end of the tube opposite the mesh filter.

6. The method of claim 2, wherein the filter device further comprises an O-ring, a gasket, or a flange slid over a step on the end of the tube adjacent the mesh filter and one or more notches on an end of the tube opposite the mesh filter.

7. The method of claim 1, wherein the filter device is inserted at a depth of between 0.25 and 5 inches below the surface of the liquid in the carrier.

8. The method of claim 1, wherein the step of collecting the fecal sample is collected with a sample collection tool containing a plunger.

9. The method of claim 8, wherein the step of collecting includes drawing back the plunger to a metered position on the sample collection tool and filling the sample collection tool with the fecal sample; and wherein the step of depositing the fecal sample includes depressing the plunger of the sample collection tool such that the fecal sample is deposited within the carrier.

10. The method of claim 1, further comprising depositing a dispersion aid into the carrier.

11. The method of claim 1, further comprising the step of centrifuging the carrier after the step of shaking.

12. The method of claim 1, further comprising the step of placing the capture filter within a reagent dispensing unit for the dispensing of reagents that will turn the collected parasitic material into fluorescent parasitic material.

13. The method of claim 12, further comprising the step of placing the capture filter within an imaging unit for imaging of the fluorescent parasitic material for analysis of the fluorescent parasitic material.

14. The method of claim 1, wherein the flotation medium has a density of between about 1.1 g/mL and about 1.2 g/mL.

15. A kit for use in analyzing a fecal sample comprising:
a. a filter device comprising a tube and a mesh filter attached to an end of said tube;
b. a sample collection tool; and
c. a fecal sample carrier.

16. The kit of claim 15, wherein the mesh filter of the filter device has a pore size of between about 20 microns and about 1,000 microns.

17. The kit of claim 15, wherein the filter device further comprises an O-ring, a gasket, or a flange secured within a groove in the tube adjacent the mesh filter and one or more notches on an end of the tube opposite the mesh filter.

18. The kit of claim 15, wherein the filter device further comprises an O-ring, a gasket, or a flange slid over a step on the end of the tube adjacent the mesh filter and one or more notches on an end of the tube opposite the mesh filter.

19. The kit of claim 15, further comprising a dispersion aid placeable within the fecal sample carrier.

20. The kit of claim 15, wherein the sample collection tool contains a plunger.
